(19) United States
(12) Patent Application Publication
(10) Pub. No.: US 2025/0221914 A1
(43) Pub. Date: Jul. 10, 2025

(54) HAIR CONDITIONER FORMULATION

(71) Applicants: Rohm and Haas Company, Collegeville, PA (US); Dow Silicones Corporation, Midland, MI (US)

(72) Inventors: Nisaraporn Suthiwangcharoen, Midland, MI (US); Lyndsay M. Leal, Spring City, PA (US); Benjamin Reiner, Collegeville, PA (US); Scott Backer, Phoenixville, PA (US)

(21) Appl. No.: 18/847,701

(22) PCT Filed: Mar. 20, 2023
(86) PCT No.: PCT/US2023/015625

§ 371 Date: Sep. 17, 2024

Related U.S. Application Data
(60) Provisional application No. 63/322,240, filed on Mar. 22, 2022.

(51) Int. Cl. A61K 8/73; A61K 8/891; A61Q 5/12

(52) U.S. Cl. CPC A61K 8/73; A61K 8/891; A61Q 5/12

(57) ABSTRACT

A hair conditioner formulation is provided, comprising: a vehicle; and a conditioning polymer, wherein the conditioning polymer is a cationic dextran polymer, comprising a dextran base polymer functionalized with morpholine groups and quaternary ammonium groups; wherein the dextran base polymer has a weight average molecular weight of 10,000 to 3,000,000 Daltons; wherein the morpholine groups are of formula (II) bound to a pendent oxygen on the dextran base polymer (II); and wherein the quaternary ammonium groups are of formula (III) bound to a pendent oxygen on the dextran base polymer (III); wherein (I) is a pendent oxygen on the dextran base polymer; wherein A and X are a divalent linking group; wherein R² is a linear or branched C₁₋₄ alkyl group; wherein R³ is a linear or branched C₈₋₂₀ alkyl group.

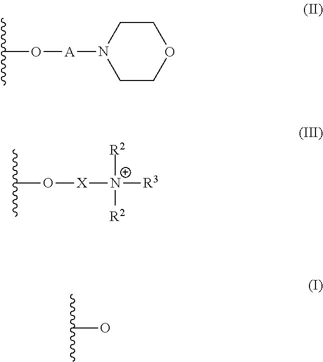

The present invention relates to a hair conditioner formulation. In particular, present invention relates to a hair conditioner formulation containing: a dermatologi acceptable vehicle; and a conditioning polymer, wherein the conditioning polymer is cationic dextran polymer, comprising a dextran base polymer functionalized with mc groups and quaternary ammonium groups; wherein the dextran base polymer has a w average molecular weight of 10,000 to 3,000,000 Daltons; wherein the morpholine g of formula (II) bound to a pendent oxygen on the dextran base polymer

[symbol]

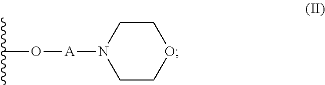

and wherein the quaternary ammonium groups are of formula (III) bound to a pendent ox the dextran base polymer

[symbol]

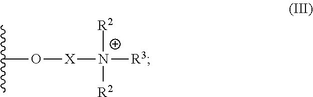

wherein

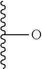 [SMILES: *O]

is a pendent oxygen on the dextran base polymer; wherein A is a di linking group; wherein X is a divalent linking group; wherein each R² is independent selected from a linear or branched C₁₋₄ alkyl group; wherein each R³ is independent; selected from a linear or branched C₈₋₂₀ alkyl group.

[symbol]

Conventional hair conditioners are popular with consumers for treating hair. based conditioning agents are the most commonly used conditioning agent in hair co formulations. However, there are growing concerns among some consumers regarding persistence and potential toxicity of certain silicone based conditioning agents or trac compounds incorporated with such silicone based conditioning agents in the environ particularly for D4 and D5 conditioners. Accordingly, there has been a growing inte the development of silicone-free alternative conditioning agents for use in hair condi formulations.

[symbol]

In U.S. Pat. No. 5,879,670, Melby et al disclose a non-silicon containing amphyolyte polymer for use as a conditioning agent for treatment of a keratin-contai substrate. In particular, Melby et al disclose novel conditioning polymer containing (meth)acrylamidopropyltrimethyl ammonium chloride, meth (acrylic acid) or 2-(meth)acrylamido-2-methylpropane sulfonic acid and, optionally, a C₁₋₂₂ alkyl (meth)acrylate and the use thereof in a cosmetically acceptable medium for the treatr keratin-containing substrate (preferably, mammalian hair; more preferably, human

[symbol]

Notwithstanding, there is a continuing need for new hair conditioning agents provide conditioning benefits. There is also a continuing need for new hair condition agents having an increased natural origin index (ISO16128) when compared with conventional hair conditioning agents.

[symbol]

The present invention provides a hair conditioner formulation, comprising: a dermatologically acceptable vehicle; and a conditioning polymer, wherein the condit polymer is a cationic dextran polymer, comprising a dextran base polymer functiona with morpholine groups and quaternary ammonium groups; wherein the dextran base polymer has a weight average molecular weight of 10,000 to 3,000,000 Daltons; whe morpholine groups are of formula (II) bound to a pendent oxygen on the dextran bas polymer and wherein the quaternary ammonium groups are of formula (III) bound tc pendent oxygen on the dextran base polymer

[symbol]

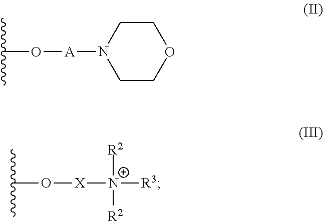

wherein

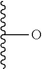 [SMILES: *O]

is a pendent oxygen on the dextran base polymer; wherein A is a di linking group; wherein X is a divalent linking group; wherein each R² is independen selected from a linear or branched C₁₋₄ alkyl group (preferably, a linear or branched [symbol] group; more preferably, a C₁₋₂ alkyl group; most preferably, a methyl group); wherein R³ is independently selected from a linear or branched C₈₋₂₀ alkyl group.

The present invention provides a hair conditioner formulation, comprising: a dermatologically acceptable vehicle; and a conditioning polymer, wherein the condit polymer is a cationic dextran polymer, comprising a dextran base polymer functiona with morpholine groups and quaternary ammonium groups; wherein the dextran base polymer has a weight average molecular weight of 10,000 to 3,000,000 Daltons; whe morpholine groups are of formula (II) bound to a pendent oxygen on the dextran bas polymer and wherein the quaternary ammonium groups are of formula (III) bound

[symbol]

pendent oxygen on the dextran base polymer; wherein

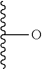 [SMILES: *O]

is a pendent oxygen dextran base polymer; wherein A is a divalent linking group; wherein X is a divalent group; wherein each R² is independently selected from a linear or branched C₁₋₄ alky (preferably, a linear or branched C₁₋₃ alkyl group; more preferably, a C₁₋₂ alkyl group preferably, a methyl group); wherein each R³ is independently selected from a linear branched C₈₋₂₀ alkyl group; and wherein the hair conditioner formulation contains l 0.01 wt %, based on weight of the hair conditioner formulation, of octamethylcyclotetrasiloxane (D4), decamethylcyclopentasiloxane (D5) and dodecamethylcyclohexasiloxane (D6).

[symbol]

The present invention provides a method of conditioning hair, comprising: S hair conditioner formulation of the present invention; and applying the hair condition formulation to hair.

[symbol]

### DETAILED DESCRIPTION

We have surprisingly found that a cationic dextran polymer, comprising a de base polymer functionalized with morpholine groups and quaternary ammonium gro wherein the dextran base polymer has a weight average molecular weight of 10,000 3,000,000 Daltons; wherein the morpholine groups are of formula (II) bound to a per oxygen on the dextran base polymer

[symbol]

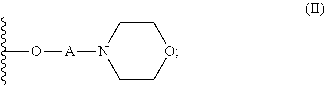

and
wherein the quaternary ammonium groups are of formula (III) bound to a pendent ox the dextran base polymer

[symbol]

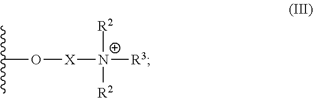

wherein

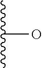 [SMILES: *O]

is a pendent oxygen on the dextran base polymer; wherein A is a di linking group; wherein X is a divalent linking group; wherein each R² is independen selected from a linear or branched C₁₋₄ alkyl group; wherein each R³ is independentl selected from a linear or branched C₈₋₂₀ alkyl group; acts as a conditioning polymer t effectively restores hydrophobicity to damaged hair and reduces the force required to treated hair, while also providing an improved natural origin index (ISO16128) wher compared with conventional hair conditioning agents.

[symbol]

Unless otherwise indicated, ratios, percentages, parts, and the like are by wei

[symbol]

As used herein, unless otherwise indicated, the phrase “molecular weight” or refers to the weight average molecular weight as measured in a conventional manner permeation chromatography (GPC) and conventional standards, such as polyethylen standards. GPC techniques are discussed in detail in Modern Size Exclusion [symbol] Chromatography, W. W. Yau, J. J. Kirkland, D. D. Bly; Wiley-Interscience, 1979, ar Guide to Materials Characterization and Chemical Analysis, J. P. Sibilia; VCH, 1988 84. Molecular weights are reported herein in units of Daltons, or equivalently, g/mo

The term “dermatologically acceptable” as used herein and in the appended c refers to ingredients that are typically used for topical application to the skin, and is i to underscore that materials that are toxic when present in the amounts typically four care compositions are not contemplated as part of the present invention.

[symbol]

The term “damaged human hair” as used herein and in the appended claims r at least one of chemically damaged human hair (e.g., human hair damaged from cher treatments such as dyeing, bleaching, perming); thermally damaged human hair (e.g. hair damaged from exposure to heat via ironing, forced drying, styling); and physica damaged human hair (e.g., human hair damaged from physical abuse such as friction curling).

[symbol]

Preferably, the hair conditioner formulation of the present invention is selecte the group consisting of a rinse off conditioner formulation and a leave on conditione formulation. More preferably, the hair conditioner formulation of the present invent rinse off conditioner formulation.

[symbol]

Preferably, the hair conditioner formulation of the present invention, comprises dermatologically acceptable vehicle (preferably, wherein the hair conditioner formul comprises 25 to 99.9 wt % (preferably, 48 to 99.85 wt %; more preferably, 79 to 99.8 most preferably, 84.5 to 99.75 wt %), based on weight of the hair conditioner formula a dermatologically acceptable vehicle); and a conditioning polymer (preferably, 0.05 wt % (preferably, 0.1 to 2 wt %; more preferably, 0.15 to 1 wt %; most preferably, 0.2 wt %), based on weight of the hair conditioner formulation, of the conditioning polyr wherein the conditioning polymer is a cationic dextran polymer, comprising a dextra polymer functionalized with morpholine groups and quaternary ammonium groups; ‘the dextran base polymer has a weight average molecular weight of 10,000 to 3,000, Daltons; wherein the morpholine groups are of formula (II) bound to a pendent oxyg dextran base polymer

[symbol]

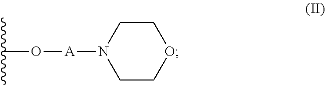

and wherein the quaternary ammonium groups are of formula (III) bound to a pendent ox the dextran base polymer

[symbol]

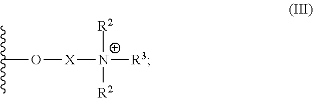

wherein

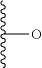 [SMILES: *O]

is a pendent oxygen on the dextran base polymer; wherein A is a di linking group (preferably, wherein A is selected from divalent alkyl groups, which n optionally be substituted with a hydroxy group, an alkoxy group and/or an ether grou preferably, wherein A is selected from the group consisting of a—(CH₂)_{y}—group and a—CH₂CH(OR⁴)CH₂—group, where y is 2 to 5 (preferably, 2 to 4; more preferably, 2 most preferably, 2) and where R⁴ is selected from the group consisting of a hydroger linear or branched C₁₋₄ alkyl group; still more preferably, wherein A is selected from group consisting of a—CH₂CH₂—group and a—CH₂CH(OH) CH₂—group; most prefera wherein A is a—CH₂CH₂—group); wherein X is a divalent linking group (preferably, X is selected from divalent alkyl groups, which may optionally be substituted with a group, an alkoxy group and/or an ether group; more preferably, wherein X is a—CH₂CH(OR⁴) CH₂—group, where R⁴ is selected from the group consisting of a hyd and a linear or branched C₁₋₄ alkyl group; most preferably, wherein X is a—CH₂CH(OH) CH₂—group); wherein each R² is independently selected from a linear branched C₁₋₄ alkyl group (preferably, a linear or branched C₁₋₃ alkyl group; more pr a C₁₋₂ alkyl group; most preferably, a methyl group); wherein each R³ is independent selected from a linear or branched C₈₋₂₀ alkyl group (preferably, a linear or branched alkyl group; more preferably, a linear or branched C₁₀₋₁₄ alkyl group; still more prefe linear or branched C₁₂ alkyl group; most preferably, a linear C₁₂ alkyl group).

[symbol]

Preferably, the hair conditioner formulation of the present invention is a liqui formulation. More preferably, the hair conditioner formulation of the present invent aqueous liquid formulation.

[symbol]

Preferably, the hair conditioner formulation of the present invention, comprises 99.9 wt % (preferably, 48 to 99.85 wt %; more preferably, 79 to 99.8 wt %; most prefe 84.5 to 99.75 wt %), based on weight of the hair conditioner formulation, of a dermatologically acceptable vehicle. More preferably, the hair conditioner formulati present invention, comprises: 25 to 99.9 wt % (preferably, 48 to 99.85 wt %; more pr 79 to 99.8 wt %; most preferably, 84.5 to 99.75 wt %), based on weight of the hair col formulation, of a dermatologically acceptable vehicle; wherein the dermatologically acceptable vehicle comprises water. Still more preferably, the hair conditioner form the present invention, comprises: 25 to 99.9 wt % (preferably, 48 to 99.85 wt %; mor preferably, 79 to 99.8 wt %; most preferably, 84.5 to 99.75 wt %), based on weight of conditioner formulation, of a dermatologically acceptable vehicle; wherein the dermatologically acceptable vehicle is selected from the group consisting of water ar aqueous C₁₋₄ alcohol mixture. Most preferably, the hair conditioner formulation of t present invention, comprises: 25 to 99.9 wt % (preferably, 48 to 99.85 wt %; more pr 79 to 99.8 wt %; most preferably, 84.5 to 99.75 wt %), based on weight of the hair col formulation, of a dermatologically acceptable vehicle; wherein the dermatologically acceptable vehicle is water.

[symbol]

Preferably, the water used in the hair conditioner formulation of the present i is at least one of distilled water and deionized water. More preferably, the water use hair conditioner formulation of the present invention is distilled and deionized.

[symbol]

Preferably, the hair conditioner formulation of the present invention, comprises to 5 wt % (preferably, 0.1 to 2 wt %; more preferably, 0.15 to 1 wt %; most preferably 0.5 wt %), based on weight of the hair conditioner formulation, of a conditioning pol wherein the conditioning polymer is a cationic dextran polymer, comprising a dextra polymer functionalized with morpholine groups and quaternary ammonium groups; “the dextran base polymer has a weight average molecular weight of 10,000 to 3,000, Daltons; wherein the morpholine groups are of formula (II) bound to a pendent oxyg dextran base polymer; and wherein the quaternary ammonium groups are of formula bound to a pendent oxygen on the dextran base polymer.

[symbol]

Preferably, the dextran base polymer has a weight average molecular weight 10,000 to 3,000,000 Daltons (preferably, 50,000 to 2,500,000 Daltons; more preferal 100,000 to 2,000,000 Daltons; still more preferably, 125,000 to 1,000,000 Daltons; r preferably, 140,000 to 500,000 Daltons). More preferably, the dextran base polymer weight average molecular weight of 10,000 to 3,000,000 Daltons (preferably, 50,00 2,500,000 Daltons; more preferably, 100,000 to 2,000,000 Daltons; still more prefer 125,000 to 1,000,000 Daltons; most preferably, 140,000 to 500,000 Daltons); and the base polymer is a branched chain dextran polymer comprising a plurality of glucose structural units; wherein 90 to 98 mol % (preferably, 92.5 to 97.5 mol %; more prefer to 97 mol %; most preferably, 94 to 96 mol %) of the glucose structural units are conr α-1,6 linkages and 2 to 10 mol % (preferably, 2.5 to 7.5 mol %; more preferably, 3 to most preferably, 4 to 6 mol %) of the glucose structural units are connected by α-1,2 α-1,3 linkages and/or α-1,4 linkages. Most preferably, the dextran base polymer has average molecular weight of 10,000 to 3,000,000 Daltons (preferably, 50,000 to 2,50 Daltons; more preferably, 100,000 to 2,000,000 Daltons; still more preferably, 125,0 1,000,000 Daltons; most preferably, 140,000 to 500,000 Daltons); and the dextran ba polymer is a branched chain dextran polymer comprising a plurality of glucose struc units; wherein 90 to 98 mol % (preferably, 92.5 to 97.5 mol %; more preferably, 93 tc mol %; most preferably, 94 to 96 mol %) of the glucose structural units are connected α-D-1,6 linkages and 2 to 10 mol % (preferably, 2.5 to 7.5 mol %; more preferably, 3 mol %; most preferably, 4 to 6 mol %) of the glucose structural units are connected by linkages according to formula I

[symbol]

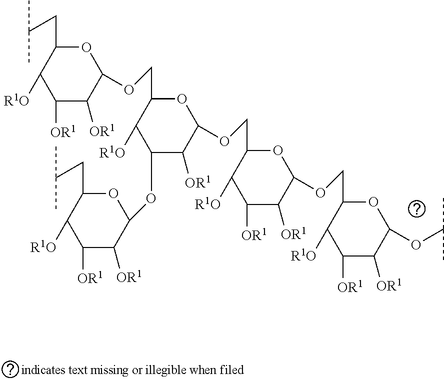

wherein R¹ is selected from a hydrogen, a C₁₋₄ alkyl group and a hydroxy C₁₋₄ alkyl and wherein the average branch off the dextran polymer backbone is 1 to 3 anhydrog units.

[symbol]

Preferably, the dextran base polymer contains less than 0.01 wt %, based on v the dextran base polymer, of alternan. More preferably, the dextran base polymer cc less than 0.001 wt %, based on weight of the dextran base polymer, of alternan. Mos preferably, the dextran base polymer contains less than the detectable limit of alterna

[symbol]

Preferably, <0.1 mol % (preferably, <0.01 mol %; more preferably, <0.001 r most preferably, <detectable limit), of the glucose structural units in the dextran bas polymer are connected by β-1,4 linkages.

[symbol]

Preferably, <0.1 mol % (preferably, <0.01 mol %; more preferably, <0.001 r most preferably, <detectable limit), of the glucose structural units in the dextran bas polymer are connected by β-1,3 linkages.

[symbol]

Preferably, the hair conditioner formulation of the present invention, comprises to 5 wt % (preferably, 0.1 to 2 wt %; more preferably, 0.15 to 1 wt %; most preferably 0.5 wt %), based on weight of the hair conditioner formulation, of a conditioning pol wherein the conditioning polymer is a cationic dextran polymer, comprising a dextra polymer functionalized with morpholine groups and quaternary ammonium groups; the morpholine groups are of formula (II) bound to a pendent oxygen on the dextran polymer and wherein the quaternary ammonium groups are of formula (III) bound tc pendent oxygen on the dextran base polymer

[symbol]

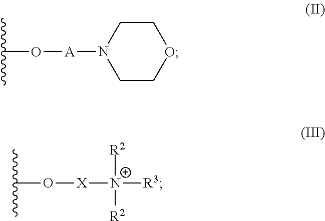

wherein

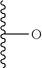 [SMILES: *O]

is a pendent oxygen on the dextran base polymer; wherein A is a di linking group (preferably, wherein A is selected from divalent alkyl groups, which n optionally be substituted with a hydroxy group, an alkoxy group and/or an ether grot preferably, wherein A is selected from the group consisting of a—(CH₂)_{y}—group and a—CH₂CH(OR⁴) CH₂—group, where y is 2 to 5 (preferably, 2 to 4; more preferably, 2 most preferably, 2) and where R⁴ is selected from the group consisting of a hydroger linear or branched C₁₋₄ alkyl group; still more preferably, wherein A is selected from group consisting of a—CH₂CH₂—group and a—CH₂CH(OH) CH₂—group; most prefera wherein A is a—CH₂CH₂—group); wherein X is a divalent linking group (preferably, X is selected from divalent alkyl groups, which may optionally be substituted with a group, an alkoxy group and/or an ether group; more preferably, wherein X is a—CH₂CH(OR⁴) CH₂—group, where R⁴ is selected from the group consisting of a hyd and a linear or branched C₁₋₄ alkyl group; most preferably, wherein X is a—CH₂CH(OH) CH₂—group); wherein each R² is independently selected from a linear branched C₁₋₄ alkyl group (preferably, a linear or branched C₁₋₃ alkyl group; more pr a C₁₋₂ alkyl group; most preferably, a methyl group); wherein each R³ is independent selected from a linear or branched C₈₋₂₀ alkyl group (preferably, a linear or branched alkyl group; more preferably, a linear or branched C₁₀₋₁₄ alkyl group; still more prefe linear or branched C₁₂ alkyl group; most preferably, a linear C₁₂ alkyl group) (prefer wherein the degree of substitution, DS (II), of the morpholine groups of formula (II) o cationic dextran polymer is 0.01 to 0.2 (preferably, 0.02 to 0.175; more preferably, 0 0.16; most preferably, 0.04 to 0.14); and wherein the degree of substitution, DS_{(III)}, 0 quaternary ammonium groups of formula (III) on the cationic dextran polymer is >0 [symbol] (preferably, 0.1 to 2; more preferably, 0.2 to 1.75; still more preferably, 0.25 to 1.5; 1 preferably, 0.3 to 1.2)). More preferably, the hair conditioner formulation of the pre invention comprises 0.05 to 5 wt % (preferably, 0.1 to 2 wt %; more preferably, 0.15 most preferably, 0.2 to 0.5 wt %), based on weight of the hair care formulation, of a conditioning polymer; wherein the conditioning polymer is a cationic dextran polym comprising a dextran base polymer functionalized with morpholine groups and quate ammonium groups; wherein the morpholine groups are of formula (IIa) bound to a p oxygen on the dextran base polymer

[symbol]

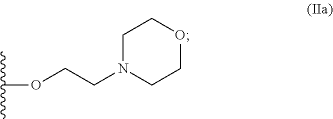 [SMILES: *OCCN1CCOCC1]

and (ii) a quaternary ammonium group of formula (IIIa) bound to a pendent oxygen on th dextran base polymer

[symbol]

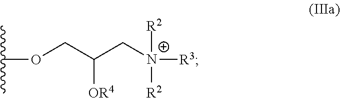

wherein

[symbol]

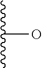 [SMILES: *O]

is a pendent oxygen on the dextran base polymer; wherein R⁴ is sel from the group consisting of a hydrogen and a linear or branched C₁₋₄ alkyl group (p R⁴ is a hydrogen); wherein each R² is independently selected from a linear or branch alkyl group (preferably, a C₁₋₃ alkyl group; more preferably, a C₁₋₂ alkyl group; most preferably, a methyl group); wherein each R³ is independently selected from a linear branched C₈₋₂₀ alkyl group (preferably, a linear or branched C₁₀₋₁₆ alkyl group; more preferably, a linear or branched C₁₀₋₁₄ alkyl group; still more preferably, a linear or b C₁₂ alkyl group; most preferably, a linear C₁₂ alkyl group); wherein the degree of sut DS (II), of the morpholine groups of formula (II) on the cationic dextran polymer is 0.1 (preferably, 0.02 to 0.175; more preferably, 0.03 to 0.16; most preferably, 0.04 to 0.1 wherein the degree of substitution, DS_{(III)}, of the quaternary ammonium groups of fo (III) on the cationic dextran polymer is >0 to 2 (preferably, 0.1 to 2; more preferably [symbol] 1.75; still more preferably, 0.25 to 1.5; most preferably, 0.3 to 1.2)). Most preferably hair conditioner formulation of the present invention comprises 0.05 to 5 wt % (prefe 0.1 to 2 wt %; more preferably, 0.15 to 1 wt %; most preferably, 0.2 to 0.5 wt %), base weight of the hair conditioner formulation, of a conditioning polymer; wherein the conditioning polymer is a cationic dextran polymer, comprising a dextran base polyr functionalized with morpholine groups and quaternary ammonium groups; wherein t morpholine groups are of formula (IIa) bound to a pendent oxygen on the dextran ba polymer; and wherein the quaternary ammonium groups are of formula (IIIa) bound pendent oxygen on the dextran base polymer; wherein each R² is a methyl group; wh each R³ is independently selected from a linear or branched C₈₋₂₀ alkyl group (prefer linear or branched C₁₀₋₁₆ alkyl group; more preferably, a linear or branched C₁₀₋₁₄ alk still more preferably, a linear or branched C₁₂ alkyl group; most preferably, a linear (group); wherein each R⁴ is a hydrogen; wherein the degree of substitution, DS (II), of morpholine groups of formula (II) on the cationic dextran polymer is 0.01 to 0.2 (pre 0.02 to 0.175; more preferably, 0.03 to 0.16; most preferably, 0.04 to 0.14); and whe degree of substitution, DS_{(III)}, of the quaternary ammonium groups of formula (III) o cationic dextran polymer is >0 to 2 (preferably, 0.1 to 2; more preferably, 0.2 to 1.7: more preferably, 0.25 to 1.5; most preferably, 0.3 to 1.2)).

[symbol]

Preferably, the deposition aid polymer has a Kjeldahl nitrogen content, TKN, 2.5 wt % (preferably, 0.55 to 2.2 wt %; more preferably, 0.6 to 2 wt %; most preferabl 1.75 wt %) measured using a Buchi KjelMaster K-375 automated analyzer, corrected volatiles and ash measured as described in ASTM method D-2364.

[symbol]

Preferably, the conditioning polymer comprises<0.001 meq/gram (preferabl 0.0001 meq/gram; more preferably, <0.00001 meq/gram; most preferably, <detecta of aldehyde functionality.

[symbol]

Preferably, the conditioning polymer comprises<0.1% (preferably, <0.01% preferably, <0.001%; most preferably, <detectable limit), of the linkages between individual glucose units in the conditioning polymer are β-1,4 linkages.

[symbol]

Preferably, the conditioning polymer comprises<0.1% (preferably, <0.01% preferably, <0.001%; most preferably, <detectable limit), of the linkages between individual glucose units in the conditioning polymer are β-1,3 linkages.

[symbol]

Preferably, the conditioning polymer comprises<0.001 meq/gram (preferabl 0.0001 meq/gram; more preferably, <0.00001 meq/gram; most preferably, <detecta of silicone containing functionality.

[symbol]

Preferably, the hair conditioner formulation of the present invention, optional further comprises at least one additional ingredient selected from the group consistin antimicrobial agent/preservative (e.g., benzoic acid, sorbic acid, phenoxyethanol, methylisothiazolinone, ethylhexyl glycerin); a rheology modifier (e.g., PEG-150 pentaerythrityl tetrastearate); a colorant; pH adjusting agent; an antioxidant (e.g., but hydroxytoluene); a humectant (e.g., glycerin, sorbitol, monoglycerides, lecithins, gly fatty alcohols, fatty acids, polysaccharides, sorbitan esters, polysorbates (e.g., Polysc Polysorbate 40, Polysorbate 60, and Polysorbate 80), diols (e.g., propylene glycol), c analogs, triols, triol analogs, cationic polymeric polyols); a wax; a foaming agent; an emulsifying agent; a colorant; a fragrance; a chelating agent (e.g., tetrasodium ethyle diamine tetraacetic acid); a preservative (e.g., benzoic acid, sorbic acid, phenoxyetha methylisothiazolinone); a bleaching agent; a lubricating agent; a sensory modifier; a sunscreen additive; a vitamin; a protein/amino acid; a plant extract; a natural ingredi bioactive agent; an anti-aging agent; a pigment; an acid; a penetrant; an anti-static ag anti-frizz agent; an antidandruff agent; a hair waving/straightening agent; a hair styli an absorbent; a conditioning agent (e.g., guar hydroxypropyltrimonium chloride, PQ 7); a slip agent; an opacifier; a pearlizing agent and a salt. More preferably, the hair conditioner formulation of the present invention, optionally, further comprises at lea additional ingredient selected from the group consisting of an antimicrobial agent/preservative (e.g., benzoic acid, sorbic acid, phenoxyethanol, methylisothiazol ethylhexyl glycerin); a rheology modifier (e.g., PEG-150 pentaerythrityl tetrastearate chelating agent (e.g., tetrasodium ethylene diamine tetraacetic acid). Most preferabl hair conditioner formulation of the present invention, optionally, further comprises a one additional ingredient selected from the group consisting of a mixture of phenoxy and methylisothiazolinone; a mixture of phenoxyethanol and ethylhexyl glycerin; PE pentaerythrityl tetrastearate; and tetrasodium ethylene diamine tetraacetic acid.

[symbol]

Preferably, the hair conditioner formulation of the present invention further c a thickener. More preferably, the hair conditioner formulation of the present inventi further comprises a thickener, wherein the thickener is selected to increase the viscos the hair conditioner formulation, preferably without substantially modifying the othe properties of the hair conditioner formulation. Preferably, the hair conditioner formu the present invention further comprises a thickener, wherein the thickener is selected increase the viscosity of the hair conditioner formulation, preferably without substan modifying the other properties of the hair conditioner formulation and wherein the th [symbol] accounts for 0 to 5.0 wt % (preferably, 0.1 to 5.0 wt %; more preferably, 0.2 to 2.5 w preferably, 0.5 to 2.0 wt %), based on weight of the hair conditioner formulation.

[symbol]

Preferably, the hair conditioner formulation of the present invention further c an antimicrobial agent/preservative. More preferably, the hair conditioner formulati present invention further comprises an antimicrobial/preservative, wherein the antimicrobial/preservative is selected from the group consisting of phenoxyethanol, ethylhexyl glycerin, benzoic acid, benzyl alcohol, sodium benzoate, DMDM hydanto 2-ethylhexyl glyceryl ether, isothiazolinone (e.g., methylchloroisothiazolinone, methylisothiazolinone) and mixtures thereof. Most preferably, the hair conditioner formulation of the present invention, further comprises an antimicrobial/preservative wherein the antimicrobial/preservative is a mixture selected from the group consistin phenoxyethanol and ethylhexyl glycerin and (b) phenoxyethanol and an isothiazolin ((more preferably, wherein the antimicrobial/preservative is a mixture selected from t consisting of (a) phenoxyethanol and ethylhexyl glycerin and (b) phenoxyethanol an methylisothiazolinone; most preferably, wherein the antimicrobial/preservative is a r of phenoxyethanol and ethylhexyl glycerin).

[symbol]

Preferably, the hair conditioner formulation of the present invention optional comprises a pH adjusting agent. More preferably, the hair conditioner formulation o present invention, further comprises a pH adjusting agent, wherein the hair conditior formulation has a pH of 4 to 9 (preferably, 4.25 to 8; more preferably, 4.5 to 7; most preferably, 4.75 to 6).

[symbol]

Preferably, the pH adjusting agent is selected from the group consisting of at of citric acid, lactic acid, hydrochloric acid, aminoethyl propanediol, triethanolamine monoethanolamine, sodium hydroxide, potassium hydroxide, amino-2-methyl-1-proj More preferably, the pH adjusting agent is selected from the group consisting of at l of citric acid, lactic acid, sodium hydroxide, potassium hydroxide, triethanolamine, amino-2-methyl-1-propanol. Still more preferably, the pH adjusting agent includes (acid. Most preferably, the pH adjusting agent is citric acid.

[symbol]

Preferably, the hair conditioner formulation of the present invention, contains wt % (preferably, <0.001 wt %; more preferably, <0.0001 wt %; most preferably, <d limit), based on weight of the hair conditioner formulation of a dermatologically acc non-silicone oil. More preferably, the hair conditioner formulation of the present in contains<0.01 wt % (preferably, <0.001 wt %; more preferably, <0.0001 wt %; mos preferably, <detectable limit), based on weight of the hair conditioner formulation o [symbol] dermatologically acceptable non-silicone oil; wherein the dermatologically acceptab non-silicone oil is selected from the group consisting of hydrocarbon oils (e.g., mine petroleum jelly, polyisobutene, hydrogenated polyisobutene, hydrogenated polydece polyisohexadecane; natural oils (e.g., caprylic and capric triglyceride, sunflower oil, oil, coconut oil, argan oil, olive oil, almond oil) and mixtures thereof.

[symbol]

Preferably, the hair conditioner formulation of the present invention contains wt % (preferably, <0.001 wt %; more preferably, <0.0001 wt %; most preferably, <d limit), based on weight of the hair conditioner formulation, of octamethylcyclotetrasi (D4) decamethylcyclopentasiloxane (D5) and dodecamethylcyclohexasiloxane (D6) combined.

[symbol]

Preferably, the hair conditioner formulation of the present invention contains wt % (preferably, <0.001 wt %; more preferably, <0.0001 wt %; most preferably, <d limit), based on weight of the hair conditioner formulation, of conditioning silicones polydimethylsiloxanes, dimethicone).

[symbol]

Preferably, the hair conditioner formulation of the present invention contains wt % (preferably, <0.001 wt %; more preferably, <0.0001 wt %; most preferably, <d limit), based on weight of the hair conditioner formulation, of silicon (Si) containing molecules.

[symbol]

Preferably, the hair conditioner formulation is selected from the group consis leave on conditioner or rinse off conditioner; wherein the hair conditioner formulatic contains<0.1 wt % (preferably, <0.001 wt %; more preferably, <0.0001 wt %; most preferably, <detectable limit), based on weight of the hair conditioner formulation, (care cleaning surfactant. More preferably, the hair conditioner formulation is selecte the group consisting of a leave on conditioner or rinse off conditioner; wherein the h conditioner formulation contains<0.1 wt % (preferably, <0.001 wt %; more preferat 0.0001 wt %; most preferably, <detectable limit), based on weight of the hair conditi formulation, of a hair care cleaning surfactant; wherein the hair cleaning surfactant is from the group consisting of alkyl polyglucosides (e.g., lauryl glucoside, coco-gluco; decyl glucoside), glycinates (e.g., sodium cocoyl glycinate), betaines (e.g., alkyl beta such as cetyl betaine and amido betaines such as cocamidopropyl betaine), taurates (sodium methyl cocoyl taurate), glutamates (e.g., sodium cocoyl glutamate), sarcosin: sodium lauroyl sarcosinate), isethionates (e.g., sodium cocoyl isethionate, sodium lau methyl isethionate), sulfoacetates (e.g., sodium lauryl sulfoacetate), alaninates (e.g., cocoyl alaninate), amphoacetates (e.g., sodium cocoamphoacetate), sulfates (e.g., so [symbol] lauryl ether sulfate (SLES)), sulfonates (e.g., sodium C₁₄₋₁₆ olefin sulfonate), succina disodium lauryl sulfosuccinate), fatty alkanolamides (e.g., cocamide monoethanolan cocamide diethanolamine, soyamide diethanolamine, lauramide diethanolamine, oled monoisopropanolamine, stearamide monoethanolamine, myristamide monoethanolar lauramide monoethanolamine, capramide diethanolamine, ricinoleamide diethanolan myristamide diethanolamine, stearamide diethanolamine, oleylamide diethanolamine tallowamide diethanolamine, lauramide monoisopropanolamine, tallowamide monoethanolamine, isostearamide diethanolamine, isostearamide monoethanolamine mixtures thereof.

[symbol]

Preferably, the method of conditioning hair of the present invention comprise selecting a hair conditioner formulation of the present invention and applying the hai conditioner formulation to the hair (preferably, mammalian hair; more preferably, ht hair; most preferably, damaged human hair). More preferably, the method of conditi hair of the present invention, comprises: selecting a hair conditioner formulation of present invention; wetting the hair (preferably, mammalian hair; more preferably, hu hair; most preferably, damaged human hair) with water; and applying the selected ha conditioner formulation to the wetted hair. Most preferably, the method of conditior of the present invention, comprises: selecting a hair conditioner formulation of the p invention; wetting the hair (preferably, mammalian hair; more preferably, human hai preferably, damaged human hair) with water; applying the selected hair conditioner formulation to the wetted hair; and then rinsing the hair with water.

[symbol]

Some embodiments of the present invention will now be described in detail i following Examples.

[symbol]

### Synthesis S1: Synthesis of Conditioning Polymer

A one ounce vial was charged with dextran polymer (2 g; Sigma-Aldrich pro D4876) and deionized water (7.4 g). A stir bar was added to the vial and the vial wa The vial was then placed on a stir plate and heated to 70° C. After the dextran was completely dissolved, a 50% aqueous sodium hydroxide solution (0.5 g) was added t contents. The vial contents continued to stir for 10 minutes at 70° C. before adding to contents a 40% aqueous solution of 3-chloro-2-hydroxypropyl-lauryl-dimethylammc chloride (0.5 g; QUAB® 342 available from SKW QUAB Chemicals). The vial cont were then stirred for 30 minutes before adding to the vial contents a 2-chloroethyl m hydrochloride salt (0.9 g). The vial contents were then stirred for 180 minutes at 70 vial contents were then cooled to room temperature. When the vial contents reached temperature, glacial acetic acid (0.4 g) was added to the vial contents. The vial conte then stirred for 10 minutes. The polymer product was then isolated by non-solvent precipitation in methanol and was characterized by 1H NMR for structural analysis t determine the degree of substitution, DS (II), of morpholino moieties and, DS_{(III)}, of dimethyldodecyl ammonium moieties reported in TABLE 2. The total Kjeldahl nitro; TKN, in the product conditioning polymers is also reported in TABLE 2.

[symbol]

### Syntheses S2-S3: Synthesis of Cationic Dextran Polymer

In Syntheses S2-S3, conditioning polymer was prepared substantially as desc Synthesis S1 but with varying reagent feeds as noted in TABLE 1. The degree of sut DS (11), of morpholino moieties and, DS (m) of the QUAB® 342 moieties in the product conditioning polymer measured by NMR is reported in TABLE 2. The total Kjeldahl nitrogen, TKN, in the product conditioning polymer is also reported in TABLE 2.

[symbol]

TABLE 1

| | Reagents (g) | | | |
|---|---|---|---|---|
| | | Chloroethyl | | |
| | | morpholine | | |
| Example | Dextran | HCl | NaOH¹ | QUAB ® 342 |
| Synthesis | 2.0 | 0.9 | 0.5 | 0.5 |
| S1 | | | | |
| Synthesis | 2.0 | 0.9 | 0.6 | 1.0 |
| S2 | | | | |
| Synthesis | 2.0 | 3.1 | 1.5 | 0 |
| S3 | | | | |

¹50% aqueous NaOH

TABLE 2

| | Degree of substitution | | TKN |
|---|---|---|---|
| Ex. | DS_{(II)} | DS_{(III)} | (wt %) |
| Synthesis S1 | 0.059 | 0.041 | 0.772 |
| Synthesis S2 | 0.126 | 0.087 | 1.469 |
| Synthesis S3 | 0.120 | 0 | 0.956 |

### Comparative Examples CF1—CF4 and Example F1-F2: Hair Conditioner Form

A hair conditioner formulation was prepared in each of Comparative Exam CF1—CF4 and Example F1-F2 having the formulation noted in TABLE 3.

[symbol]

TABLE 3

| | CF1 | CF2 | CF3 | CF4 | F1 |
|---|---|---|---|---|---|
| Ingredient INCI name | wt % | wt % | wt % | wt % | wt % |
| Deionized water | q.s. 100 | | | | |
| Hydroxyethyl cellulose¹ | 1.4 | 1.4 | 1.4 | 1.4 | 1.4 |
| Tetrasodium EDTA² | 0.2 | 0.2 | 0.2 | 0.2 | 0.2 |
| Cetearyl alcohol³ | 1.0 | 1.0 | 1.0 | 1.0 | 1.0 |
| Glyceryl stearate (and) PEG-100 | 1.0 | 1.0 | 1.0 | 1.0 | 1.0 |
| stearate⁴ | | | | | |
| Unmodified branched chain dextran⁵ | — | 0.3 | — | — | — |
| Diethylaminoethyl dextran⁶ | — | — | 0.3 | — | — |
| Polymer product of Synthesis S1 | — | — | — | — | 0.3 |
| Polymer product of Synthesis S2 | — | — | — | — | — |
| Polymer product of Synthesis S3 | — | — | — | 0.3 | — |
| Phenoxyethanol and Meth- | 0.5 | 0.5 | 0.5 | 0.5 | 0.5 |
| ylisothiazolinone⁷ | | | | | |

¹available from The Dow Chemical Company under tradename Cellosize ™ PCG-1
²available from The Dow Chemical Company under tradename Versene ™ 220
³available from Croda Inc. under tradename Crodacol ™ CS50
⁴available from Croda Inc. under tradename Arlacel ™ 165
⁵available from Sigma Aldrich under catalog number D4876
⁶available from Sigma Aldrich under catalog number D9885
⁷preservative available from The Dow Chemical Company under tradename Neolo

### Hair Conditioning Performance

Studies to evaluate ease of wet and dry combing of hair treated with a rinse o conditioner formulation of Comparative Examples CF1—CF4 and Example F1 wer performed as follows. Bleached Caucasian hair (8 hour bleached hair from Internati Importers) was used for testing the conditioners. Each tress weighed 2 grams. Each was rinsed for 30 seconds under a stream of 40° C. tap water. Using a pipette, 0.4 gr solution containing nine percent of sodium lauryl sulfate was applied and lathered th each tress for 30 seconds. The tresses were then rinsed for 1 minute under running v Excess water was removed from the tresses by passing each tress between the index middle fingers of the hand. The tresses were then treated with a rinse off conditione formulation of Comparative Examples CF1—CF4 and Example F1-F2 at 0.4 g formulation/g of hair by massaging the formulation into the wet/damp hair for 1 min tresses were rinsed for 30 seconds under running water and dried overnight at room temperature.

[symbol]

An INSTRON Model 3342 Single Column Tension running BlueHill 2 softw also used for determining conditioning performance by the ease of wet combing and of dry combing. The test employed an INSTRON strain gauge, which was equipped measure the force required to comb the hair. The conditioning performance was bas ability of the rinse off conditioner formulation, to reduce the force required to comb with the INSTRON strain gauge. The force was reported as an Average Combing L (ACL). The lower the number of the ACL value, the better the conditioning effect ir by the rinse off conditioner formulation tested.

[symbol]

According to the INSTRON wet combing method, hair was first wetted by di into distilled water, and then the hair was detangled by combining the tress three tim tress was then retangled by dipping in distilled water three times. Excess water was by passing the tress through the index and middle fingers of the hand twice. The tres placed on a hanger and INSTRON combed. An average wet combing force from thr tresses was measured for each rinse off conditioner formulation. The average wet cc results are provided in TABLE 4.

[symbol]

According to the INSTRON dry combining method, dry hair was detangled t combining the tress 3 times. Then the hair was retangled by swirling the tress clocky times and swirling it counter clockwise 3 times. The tress was then placed on a hang INSTRON combed. An average dry combining force from three tresses was measur each rinse off conditioner formulation. The average dry combining results are provi TABLE 4.

[symbol]

TABLE 4

| | ACL (kgf) | | |
|---|---|---|---|
| Rinse off Conditioner | Dry | Wet | Dry + Wet |
| Comparative Example CF1 | 0.0593 | 0.5360 | 0.5953 |
| Comparative Example CF2 | 0.0969 | 0.7067 | 0.8036 |
| Comparative Example CF3 | 0.0239 | 0.2863 | 0.3102 |
| Comparative Example CF4 | 0.0786 | 0.3033 | 0.3819 |
| Example F1 | 0.0229 | 0.1095 | 0.1324 |
| Example F2 | 0.0261 | 0.1033 | 0.1294 |

### Hair Hydrophobicity

Hair conditioner formulation prepared according to each of Comparative Ex CF1—CF4 and Example F1-F2 were tested on two separate 3 g hair samples (8 hour Caucasian Hair from International Hair Importers, Inc.). The hair samples were first with water for 30 seconds. Then a 9% w/w aqueous solution of sodium lauryl sulfat massaged into the hair samples for 30 seconds. Then the hair samples were rinsed w for 60 seconds. The hair samples were then treated with the rinse off hair conditione dosage of 0.4 g/g or hair and massaged onto the hair for 30 seconds. The hair sampl then rinsed with water for 30 seconds and dried before hydrophobicity testing.

[symbol]

To measure hydrophobicity of the hair, the tresses were combed straight and tightly on both ends with a holder. Ten 30 μL drops of water were placed at differen locations on each tress from the root to the tip and a restored hydrophobicity score of was applied for each hair conditioner formulation as reported in TABLE 5. A score o means the water droplets were observed to dissipate into the hair in less than 1 minut application. A score of 2 means the water droplets were observed to remain on the h least 1 minute but less than 2 minutes after application before dissipating into the hai score of 3 means that the water droplets were observed to remain on the hair for at l minutes but less than 5 minutes after application before dissipating into the hair. A s means the water droplets were observed to remain on the hair for at least 5 minutes t than 10 minutes after application before dissipating into the hair. A score of 5 mean: water droplets were observed to remain on the hair for at least 10 minutes after appli before dissipating into the hair.

[symbol]

TABLE 5

| Hair conditioner formulation | Restored hydrophobicity score |
|---|---|
| Comparative Example CF1 | 1 |
| Comparative Example CF2 | 1 |
| Comparative Example CF3 | 5 |
| Comparative Example CF4 | 1 |
| Example F1 | 5 |
| Example F2 | 3 |

We claim:

1. A hair conditioner formulation, comprising:
a dermatologically acceptable vehicle; and
a conditioning polymer, wherein the conditioning polymer is a cationic dextr polymer, comprising a dextran base polymer functionalized with morpholine groups quaternary ammonium groups; wherein the dextran base polymer has a weight avera molecular weight of 10,000 to 3,000,000 Daltons; wherein the morpholine groups ar formula (II) bound to a pendent oxygen on the dextran base polymer 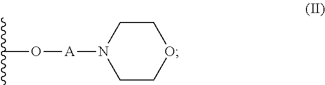
and wherein the quaternary ammonium groups are of formula (III) bound to a pendent ox the dextran base polymer 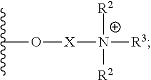
wherein 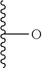 [SMILES: *O]
is a pendent oxygen on the dextran base polymer; wherein A is a di linking group; wherein X is a divalent linking group; wherein each R² is independen selected from a linear or branched C₁₋₄ alkyl group; wherein each R³ is independentl selected from a linear or branched C₈₋₂₀ alkyl group.

[symbol]

2. The hair conditioner formulation of claim 1, wherein the hair conditic formulation is selected from the group consisting of a leave on conditioner and a rins conditioner.

[symbol]

3. The hair conditioner formulation of claim 2, wherein the conditioning has a Kjeldahl nitrogen content corrected for ash and volatiles, TKN, of 0.5 to 2.5 w

[symbol]

4. The hair conditioner formulation of claim 3, wherein the hair conditic formulation contains less than 0.01 wt %, based on weight of the hair conditioner for of octamethylcyclotetrasiloxane (D4), decamethylcyclopentasiloxane (D5) and dodecamethylcyclohexasiloxane (D6) combined.

[symbol]

5. The hair conditioner formulation of claim 4, wherein the hair conditic formulation contains less than 0.01 wt %, based on weight of the hair conditioner for of a dermatologically acceptable oil.

[symbol]

6. The hair conditioner formulation of claim 5,
wherein the morpholine groups are of formula (II) are of formula (IIa) 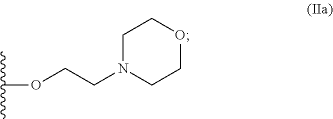 [SMILES: *OCCN1CCOCC1]
and wherein the quaternary ammonium groups are of formula (III) of formula (IIIa) 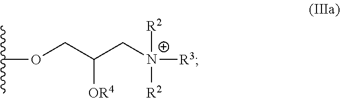
wherein each R⁴ is independently selected from the group consisting of a hyd and a linear or branched C₁₋₄ alkyl group.

[symbol]

7. The hair conditioner formulation of claim 6, wherein R² is a methyl g wherein R³ is a linear or branched C₁₂ alkyl group; and wherein R⁴ is a hydrogen.

[symbol]

8. The hair conditioner formulation of claim 7, wherein the degree of substitution, DS (1), of the morpholine groups of formula (II) on the cationic dextran] is 0.01 to 0.2; and wherein the degree of substitution, DS_{(III)}, of the quaternary ammc groups of formula (III) on the cationic dextran polymer is >0 to 2.

[symbol]

9. The hair conditioner formulation of claim 8, further comprising at lea additional ingredient selected from the group consisting of an antimicrobial agent/preservative; a rheology modifier; a soap; a colorant; pH adjusting agent; an antioxidant; a humectant; a wax; a foaming agent; an emulsifying agent; a colorant; fragrance; a chelating agent; a preservative; a bleaching agent; a lubricating agent; a modifier; a sunscreen additive; a vitamin; a protein/amino acid; a plant extract; a nat ingredient; a bioactive agent; an anti-aging agent; a pigment; an acid; a penetrant; an anti-static agent; an anti-frizz agent; an antidandruff agent; a hair waving/straightenin a hair styling agent; an absorbent; a hard particle; a soft particle; a conditioning agen agent; an opacifier; a pearlizing agent and a salt.

[symbol]

10. A method of conditioning hair, comprising:
selecting a hair conditioner formulation according to claim 1; and
applying the hair conditioner formulation to hair. [symbol]
